(19) United States
(12) Patent Application Publication
(10) Pub. No.: US 2013/0172563 A1
(43) Pub. Date: Jul. 4, 2013

(54) LENALIDOMIDE SOLVATES AND PROCESSES

(71) Applicants: Srinivasulu Rangineni, Mahaboob Nagar (IN); Nagakiran Duggirala, Krishna (IN); Vamsi Krishna Mudapaka, Khammam (IN); Rajasekhar Kadaboina, Hyderabad (IN); Veerender Murki, Hyderabad (IN); Amarendhar Manda, Hyderabad (IN); Venkata Rao Badisa, Krishna (IN); Naresh Vemula, Nalgonda (IN); Rama Seshagiri Rao Pulla, Hyderabad (IN)

(72) Inventors: Srinivasulu Rangineni, Mahaboob Nagar (IN); Nagakiran Duggirala, Krishna (IN); Vamsi Krishna Mudapaka, Khammam (IN); Rajasekhar Kadaboina, Hyderabad (IN); Veerender Murki, Hyderabad (IN); Amarendhar Manda, Hyderabad (IN); Venkata Rao Badisa, Krishna (IN); Naresh Vemula, Nalgonda (IN); Rama Seshagiri Rao Pulla, Hyderabad (IN)

(73) Assignees: Dr. Reddy's Laboratories, Inc., Bridgewater, NJ (US); Dr. Reddy's Laboratories Ltd., Hyderabad (IN)

(21) Appl. No.: 13/572,287

(22) Filed: Aug. 10, 2012

Related U.S. Application Data
(62) Division of application No. 13/109,232, filed on May 17, 2011, now abandoned.
Application No. 13/109,232 is a continuation of application No. PCT/US2009/036784, filed on Mar. 11, 2009.
(60) Provisional application No. 61/142,520, filed on Jan. 5, 2009.
(60) Provisional application No. 61/154,584, filed on Feb. 23, 2009.

(30) Foreign Application Priority Data
Nov. 17, 2008 (IN) 2829/CHE/2008
Dec. 29, 2008 (IN) 3309/CHE/2008

(51) Int. Cl. C07D 401/04

(52) U.S. Cl. CPC C07D 401/04
USPC 546/201

(57) ABSTRACT

The present application relates to lenalidomide salts and solvates, and processes for the preparation thereof.

### INTRODUCTION

Aspects of the present application relate to lenalidomide salts and solvates that are useful for making pharmaceutical dosage forms. Other aspects of the present application relate to processes for the preparation of lenalidomide salts and solvates.

The compound having the adopted name “lenalidomide” has a chemical name 3-(4-amino-1-oxo 1,3-dihydro-2H-isoindol-2-yl)piperidine-2,6-dione and is structurally represented by Formula I.

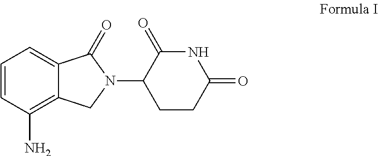 [SMILES: Nc1cccc2c1CN(C1CCC(=O)NC1=O)C2=O]

Lenalidomide, a thalidomide analogue, was initially intended for use as a treatment for multiple myeloma, for which thalidomide is an accepted therapeutic modality, but it has also shown efficacy in the hematological disorders known as the myelodysplastic syndromes (MDS). The exact mechanism of the immuno-modulatory drugs (e.g. thalidomide, CC-4047/actimid and lenalidomide) is not known. Apart from interfering with the immune system, they are also found to be active for angiogenesis. With myelodysplastic syndromes, the encouraging results of lenalidomide were also obtained in patients with deletion 5q cytogenetic abnormality (a chromosomal abnormality).

Lenalidomide was approved by the U.S. Food and Drug Administration on Dec. 27, 2005 for treating patients with low or intermediate-1 risk MDS with 5q—with or without additional cytogenetic abnormalities.

The drug is commercially marketed in products sold by Celgene Corporation under the brand name REVLIMID™ in the form of capsules having the strengths 5 mg, 10 mg, 15 mg, and 25 mg.

Muller et al., in U.S. Pat. No. 5,635,517, disclose substituted 1-oxo-2-(2,6-dioxopiperidin-3-yl)isoindolines derivatives, pharmaceutical compositions containing these compounds, and their use in the treatment of cancer. It also discloses a process for the preparation of these compounds, which involves hydrogenation of a nitro group to an amine group, using palladium on carbon in 1,4-dioxane solvent.

Muller et al., in U.S. Pat. Nos. 6,281,230 and 5,798,368, disclose a process for the preparation of (S)-3-(1-oxo-4-aminoisoindolin-2-yl)piperidine-2,6-dione by hydrogenating (S)-3-(1-oxo-4-nitroisoindolin-2-yl)piperidine-2,6-dione using 10% Pd/C in methanol, followed by slurrying in hot ethyl acetate.

Muller et al., in U.S. Patent Application Publication No. 2006/0052609, disclose another process for the preparation of lenalidomide. The process involves the hydrogenation of (S)- or racemic 3-(4-nitro-1-oxo-1,3-dihydroisoindol-2-yl)-piperidine-2,6-dione using 10% Pd on C in methanol to form (S)- or racemic 3-(4-amino-1-oxo-1,3-dihydro-2H-isoindol-2-yl)piperidine-2,6-dione.

Palle et al., in Indian Application No. 047/CHE/2006 which was published on Nov. 23, 2007, disclose a process for the preparation of lenalidomide comprising hydrogenating 3-(4-nitro-1-oxo-1,3-dihydroisoindol-2-yl)-piperidine-2,6-dione using 10% Pd on carbon in a mixture of solvents comprising methanol and N,N-dimethylformamide, to provide lenalidomide.

Chen et al., in International Application Publication No. WO 2005/023192, disclose polymorphic forms of lenalidomide, designated as forms A, B, C, D, E, F, G, and H. Particularly, Form C and Form D are solvated forms of lenalidomide. Form C is obtained from evaporation, slurries and slow cooling in acetone solvent systems, contains about 0.497 moles of acetone (is a hemisolvate of acetone) and exhibits two endotherms by DSC at about 150° C. and 269° C. Another solvated form of lenalidomide, which is designated Form D, is a crystalline solid solvated both with water and acetonitrile, is prepared by evaporation from acetonitrile solvent systems and exhibits two endotherms in DSC at about 122° C. and about 270° C. Further, the publication also mentions Form E as a solvated material, since it typically loses about 10.58% volatile matter on heating to about 125° C. and exhibits endotherms in DSC analysis at about 99° C., 161° C., and 269° C. Form E was obtained by slurrying lenalidomide in water and by slow evaporation from an acetone:water (ratio about 9:1) solvent system. Further, this publication mentions Form E as a dihydrated, crystalline material. Chen et al. have also concluded that “In aqueous solvent systems, Form E appears to be the most stable form.” Desolvation experiments performed on Form E show that upon heating at about 125° C. for about five minutes, Form E can convert to Form B, which is a hemihydrate form. Further, the publication discloses pharmaceutical compositions comprising the various crystalline forms of lenalidomide and mixtures of crystalline forms having greater than 50% crystallinity.

There remains a need for new salts and solvates which are not only useful in the preparation of pharmaceutical compositions but also have industrially feasible processes.

### SUMMARY

Aspects of the present invention relate to lenalidomide salts and solvates, and process for the preparation thereof.

In an aspect, the present invention provides processes for preparing lenalidomide solvates, an embodiment comprising:

a) reducing 3-(4-nitro-1-oxo 1,3-dihydroisoindol-2-yl)piperidine-2,6-dione of formula II,

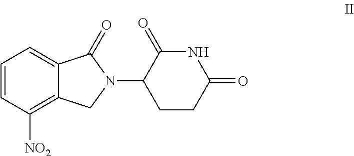 [SMILES: O=C1CCC(N2Cc3c(cccc3[N+](=O)[O-])C2=O)C(=O)N1]

with a catalyst in the presence of an acid to give an acid addition salt of lenalidomide of formula III,

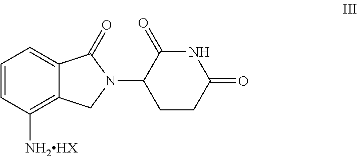

wherein HX is an acid; and

b) treating the acid addition salt of lenalidomide of formula II with a base in the presence of a solvent to provide a lenalidomide solvate.

In an embodiment, there is provided a process for the preparation of a lenalidomide solvate comprising reacting a methanesulfonate salt of lenalidomide with a base in the presence of a solvent.

In another aspect, the present invention provides solvates of lenalidomide including an N,N-dimethylformamide (DMF) solvate and a dimethylsulfoxide (DMSO) solvate.

In an embodiment, the present invention provides a process for preparing an N,N-dimethylformamide solvate of lenalidomide, comprising:

a) providing a solution of a methanesulfonate salt of lenalidomide in a solvent;

b) adding a base to the solution; and

c) isolating a DMF solvate of lenalidomide.

In another embodiment, the present invention provides a process for preparing a dimethylsulfoxide (DMSO) solvate of lenalidomide, which process comprises similar steps, but substituting dimethylsulfoxide for the N,N-dimethylformamide above.

In yet another aspect, the present invention also provides a process for preparing a solvate of lenalidomide, comprising isolating a solid from a solution of lenalidomide.

In one embodiment, the present invention also includes a process for preparing a solvate of lenalidomide, which process comprises slurrying lenalidomide in a mixture of an organic solvent and a co-solvent for a period of time sufficient to crystallize a solvate of lenalidomide. Examples of organic solvents include N,N-dimethylformamide, N,N-dimethylacetamide, dimethylsulfoxide, and mixtures thereof.

The solvate forms of lenalidomide produced according to the processes of the present invention are sufficiently stable and useful for making pharmaceutically acceptable dosage forms for the treatment of diseases including, but not limited to, multiple myeloma.

### BRIEF DESCRIPTION OF THE DRAWINGS

FIG. 1 is an X-ray powder diffraction (XRPD) pattern of an N,N-dimethylformamide solvate of lenalidomide, prepared according to Example 2.

FIG. 2 is a differential scanning calorimetry (DSC) curve of an N,N-dimethylformamide solvate of lenalidomide, prepared according to Example 2.

FIG. 3 is a thermogravimetric analysis (TGA) curve of an N,N-dimethylformamide solvate of lenalidomide, prepared according to Example 2.

FIG. 4 is an XRPD pattern of an N,N-dimethylformamide solvate of lenalidomide, prepared according to Example 6.

FIG. 5 is a TGA curve of an N,N-dimethylformamide solvate of lenalidomide, prepared according to Example 6.

FIG. 6 is an XRPD pattern of a dimethylsulfoxide solvate of lenalidomide, prepared according to Example 10.

FIG. 7 is a TGA curve of a dimethylsulfoxide solvate of lenalidomide, prepared according to Example 10.

### DETAILED DESCRIPTION

As set forth herein, an aspect of the present invention relates to lenalidomide salts and solvates, and processes for preparation thereof.

Lenalidomide and its solvates may be described by reference to patterns, spectra, curves, or other graphical data as “substantially” shown or depicted in a figure, or by one or more data points. It will be appreciated that patterns, spectra, and other graphical data can be shifted in their positions, relative intensities, or other values due to a number of factors known to those of skill in the art. For example, in the crystallographic and powder X-ray diffraction arts, shifts in peak positions or the relative intensities of one or more peaks of a pattern can occur because of, without limitation: the equipment used, the sample preparation protocol, preferred packing and orientations, the radiation source, operator error, method and length of data collection, and the like. However, those of ordinary skill in the art should be able to compare the figures herein with a pattern generated of an unknown form of, in this case, lenalidomide, and confirm its identity as one of the forms disclosed and claimed herein. The same holds true for other techniques which may be reported herein.

In addition, where a reference is made to a figure, it is permissible to, and this document includes and contemplates, the selection of any number of data points illustrated in the figure that uniquely define that crystalline form, salt, solvate, and/or optical isomer, within any associated and recited margin of error, for purposes of identification.

All percentages and ratios used herein are expressed by weight of the total composition and all measurements made are at about 25° C. and about normal pressure, unless otherwise designated. All temperatures are in degrees Celsius unless specified otherwise. As used herein, “comprising” (open ended) includes the elements recited, or their equivalent in structure or function, plus any other element or elements which are not recited. The terms “having” and “including” are also to be construed as open ended. As used herein, “consisting essentially of” means that the invention may include ingredients in addition to those recited in the claim, but only if the additional ingredients do not materially alter the basic and novel characteristics of the claimed invention. All ranges recited herein include the endpoints, including those that recite a range “between” two values. Whether so indicated or not, all values recited herein are approximate as defined by the circumstances, including the degree of expected experimental error, technique error, and instrument error for a given technique used to measure a value.

The terms “slurrying solvent” or “slurrying mixture” mean a solvent or solvent mixture used to form a suspension with a target compound.

The term “solvate” means that the solvent is bound with a target compound in a reproducible molar ratio, such as 0.5:1 or 1:1.

In one aspect, the present invention provides processes for preparing lenalidomide solvates, an embodiment comprising:

a) reducing 3-(4-nitro-1-oxo 1,3-dihydroisoindol-2-yl)piperidine-2,6-dione of Formula II,

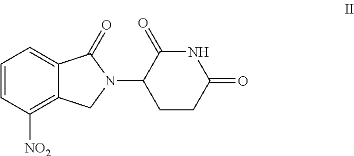 [SMILES: O=C1CCC(N2Cc3c(cccc3[N+](=O)[O-])C2=O)C(=O)N1]

with a catalyst in the presence of an acid, to form an acid addition salt of lenalidomide of formula III,

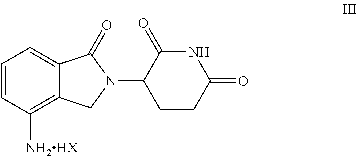

wherein HX is an acid; and

b) treating the acid addition salt of lenalidomide of formula III with a base in the presence of a solvent to provide a lenalidomide solvate.

The individual steps are separately described below.

Step a) involves reducing 3-(4-nitro-1-oxo 1,3-dihydroisoindol-2-yl)piperidine-2,6-dione of Formula II with a catalyst in the presence of an acid and a solvent to give an acid addition salt of lenalidomide having formula III, wherein HX is an acid.

The reduction of the compound of formula II may be carried out using various catalysts and in the presence of a hydrogen source. Suitable catalysts which may be used include, without limitation thereto: metal catalysts such as palladium, platinum, nickel, iridium, ruthenium, and the like in the presence of carbon or another support; a transition metal catalyst in combination with an acid such as iron/HCl, Zn/HCl, Sn/HCl, Zn/acetic acid, or Zn/ammonium formate; Raney nickel; and the like. A catalyst may be a chemical reducing agent such as stannous chloride (SnCl₂), ferric chloride (FeCl3), zinc, in the presence of an acid like acetic acid or hydrochloric acid, or a base like hydrazine. An example of a catalyst is palladium (Pd) on carbon.

The concentrations of palladium on the support, such as carbon, that can be used for the hydrogenation reaction may range from about 1 to about 30% w/w, or about 5 to 10%, or about 10%.

For example, the quantity of 10% Pd on carbon that is used in the reaction of step a) may range from about 0.05 to 0.15 grams, per gram of 3-(4-nitro-1-oxo 1,3-dihydroisoindol-2-yl)piperidine-2,6-dione of Formula II.

Acids that may be used in the hydrogenation reaction include inorganic acids and organic acids, such as but not limited to: organic acids like alkyl- and aryl-sulfonic acids such as methane sulfonic acid, and formic acid, acetic acid, trifluoroacetic acid, or their salts; and inorganic acids such as hydrochloric acid, sulfuric acid, phosphoric acid, and the like. The quantity of acid is used for the reduction of Formula II may range from about 0.5 to about 2.5 molar equivalents, or about 1 to 2 molar equivalents, per equivalent of the compound of Formula I.

The reduction of the compound of Formula II may be carried out in the presence of a solvent to provide an acid addition salt of lenalidomide. The solvents that may be used in the hydrogenation reaction include, but are not limited to: water; alcohols like methanol, ethanol, n-propanol, isopropyl alcohol, n-butanol, and the like; ketonic solvents like acetone, ethyl methyl ketone, methyl isobutyl ketone and the like; N,N-dimethylformamide (DMF); N,N-dimethylacetamide; dimethylsulfoxide (DMSO); and mixtures thereof. For example, water or methanol may be used as the solvent in the hydrogenation reaction.

The quantity of solvent used for the hydrogenation reaction is less than about 50 times the weight of the compound of Formula II, and may also depend on the solvent selected.

The reaction of step a) may be carried out at temperatures ranging from about 20° C. to about 60° C., or about 25° C. to about 35° C.

After completion of the reaction, the acid addition salt of lenalidomide of formula III may optionally be isolated or may be converted in situ to lenalidomide solvate. In an embodiment, the reaction mixture may be filtered and concentrated to an extent where the precipitation of solid begins from the solution. Generally, the concentration procedure may be terminated when the quantity of solvent becomes less than about 15 mL per gram of 3-(4-nitro-1-oxo 1,3-dihydroisoindol-2-yl)piperidine-2,6-dione of Formula II. The reaction mixture may be maintained further at temperatures lower than the concentration temperatures such as, for example, below about 40° C., for a period of time as required for isolation of the acid addition salt of lenalidomide. The exact cooling temperatures and times required for a desired extent of crystallization can be readily determined by a person skilled in the art, and may also depend on parameters such as concentrations and temperatures of the solution or slurry. The obtained acid addition salt of lenalidomide may be purified further using suitable purification techniques such as recrystallization, slurrying in a solvent or mixture of solvents, or using solvent and anti-solvent techniques and the like.

Step b) involves treating the acid addition salt of lenalidomide of formula III of step (a) with a base in the presence of a solvent to provide the lenalidomide solvate.

The process involves providing a solution of acid addition salt of lenalidomide in a solvent, combining a base with the solution, and precipitating a solid solvate from the solution, using suitable techniques.

The solution of acid addition salt of lenalidomide can be obtained by dissolving an acid addition salt of lenalidomide in a solvent, optionally in the presence of a base.

The acid addition salt of lenalidomide can be any salt of lenalidomide obtained from a previous processing step. Any form of acid addition salt of lenalidomide is acceptable, such as any crystalline or amorphous form. In an embodiment, the acid addition salt is an alkyl- or aryl-sulfonate salt of lenalidomide.

The solution of acid addition salt of lenalidomide of formula III may be obtained in a single solvent or in a combination of co-solvents. Useful solvents include, but are not limited to, N,N-dimethylformamide (DMF), dimethylsulfoxide (DMSO) and N,N-dimethylacetamide (DMA). An optional co-solvent may be any of, without limitation thereto: a hydrocarbon solvent, such as, for example, toluene, xylene, n-hexane, n-heptane, and cyclohexane; an ester, such as, for example, ethyl acetate, n-propyl acetate, n-butyl acetate, and t-butyl acetate; an alcohol, such as, for example, methanol, ethanol, and isopropyl alcohol; an ether, such as, for example, 1,4-dioxane, diethyl ether, tetrahydrofuran, diisopropyl ether, and methyl t-butyl ether; a nitrile solvent, such as, for example, propionitrile; a halogenated solvent, such as, for example, dichloromethane, ethylene dichloride, and chloroform; a ketone, such as, for example, ethyl methyl ketone, methyl isobutyl ketone, and the like; and mixtures thereof or their combinations with water in various proportions. If a solvent is used in combination with a co-solvent, the amount of co-solvent in a solvent mixture may range from about 5% to about 95%, by volume, or may depend on the solvents used.

The solution can be prepared at any temperatures up to the boiling point of the solvent, which may range from about 20° C. to about 180° C. The amount of solvent used for preparing the solution may be readily determined by the person skilled in the art and may also depend on the temperature of dissolution.

The solution can be optionally treated with an agent such as activated charcoal to enhance the color of the compound and then typically will be filtered through an inert medium, such as through a bed of flux-calcined diatomaceous earth (e.g., HYFLO), to remove the carbon.

The solution can optionally be filtered by passing through paper, glass fiber, or other membrane material, or a clarifying agent, such as CELITE. Depending upon the equipment used and the concentration and temperature of the solution, the filtration apparatus may need to be heated to avoid premature crystallization.

Suitable bases that may be used include, but not limited to: organic bases such as, for example, pyridine, imidazole, N-methylmorpholine, alkyl amines such as triethylamine, methylamine, isopropylamine, diisopropylethylamine, and the like; and inorganic bases such as, for example, ammonia, sodium hydroxide, potassium hydroxide, lithium hydroxide, sodium carbonate, potassium carbonate, sodium bicarbonate, potassium bicarbonate, and the like.

The quantity of base, which is used in the present invention, may range from about 0.5 to about 2.5 molar equivalents, or about 1 molar equivalent, per equivalent of an acid addition salt of lenalidomide.

The base is added to the solution at a temperature of about 20° C. to about 60° C., and maintained for a suitable period of time to provide lenalidomide solvate.

A solid lenalidomide solvate may be precipitated or crystallized from the solution using suitable techniques, such as, for example, cooling for a sufficient period of time to effect the desired extent of crystallization, partial removal of the solvent from the mixture, seeding, adding an anti-solvent to the solution, or a combination thereof, as desired.

For example, lenalidomide solvate may be precipitated or crystallized by cooling the solution for a period of about 5 minutes to 2 hours, or longer, to initiate and enhance the crystallization. Suitable temperatures for crystallization may range from about 0° C. to about 50° C.

Lenalidomide solvate that separates from the solution can be recovered using conventional, suitable methods. For example, lenalidomide solvate may be isolated with filtration by gravity or by suction, centrifugation, decantation, and the like. After isolation, the solid may optionally be washed with a suitable solvent such as DMF or DMSO, optionally in combination with a co-solvent, when desired.

Examples of solvates of lenalidomide that can be prepared include, but are not limited to, an N,N-dimethylformamide solvate of lenalidomide, a dimethylsulfoxide solvate of lenalidomide, and an N,N-dimethylacetamide solvate of lenalidomide.

An example of an overall process of the present invention is represented in Scheme 1.

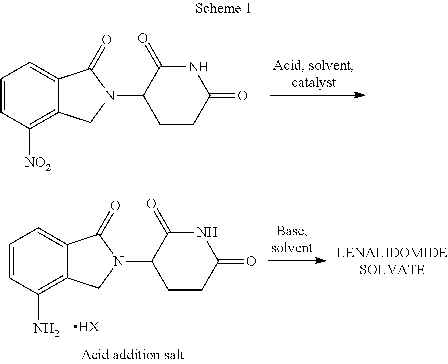

The present invention makes the use of an additional purification step optional, and introduces the use of an acid and base to minimize or avoid the formation of impurities and allow the process to be reproducible and suitable for industrial scale use. This improvement of the present invention will reduce the overall cost and enhance the reaction throughput to produce substantially pure lenalidomide.

In one embodiment, there is provided a process for the preparation of lenalidomide solvate comprising reacting a methanesulfonate salt of lenalidomide with a base, in the presence of solvent.

In an aspect, the present invention provides a solvate of lenalidomide with N,N-dimethylformamide (DMF) solvate, and a solvate of lenalidomide with dimethylsulfoxide (DMSO).

In an embodiment, the present invention provides a dimethylformamide solvate of lenalidomide, characterized by any one or more of XRPD, DSC, and TGA.

A dimethylformamide solvate of lenalidomide of the present invention may be characterized by any one or more of:

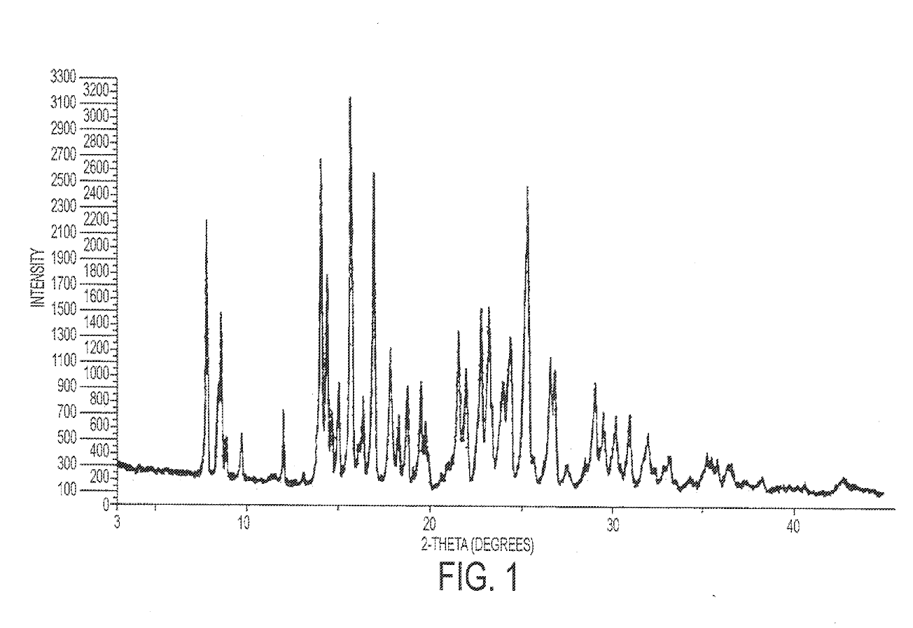

(a) An XRPD pattern substantially in accordance with FIG. 1.

(b) An XRPD pattern having characteristic peaks at about 7.9, 8.5, 8.7, 12.1, 14.1, 14.5, 15.1, 15.8, 17.0, 17.9, 18.8, 19.6, 21.6, 22.0, 22.8, 23.3, 24.0, 24.4, 25.4, 26.6, and 26.9, ±0.2 degrees 2-theta.

(c) An XRPD pattern having characteristic peaks at about 17.0, 23.3, and 25.4, ±0.2 degrees 2-theta.

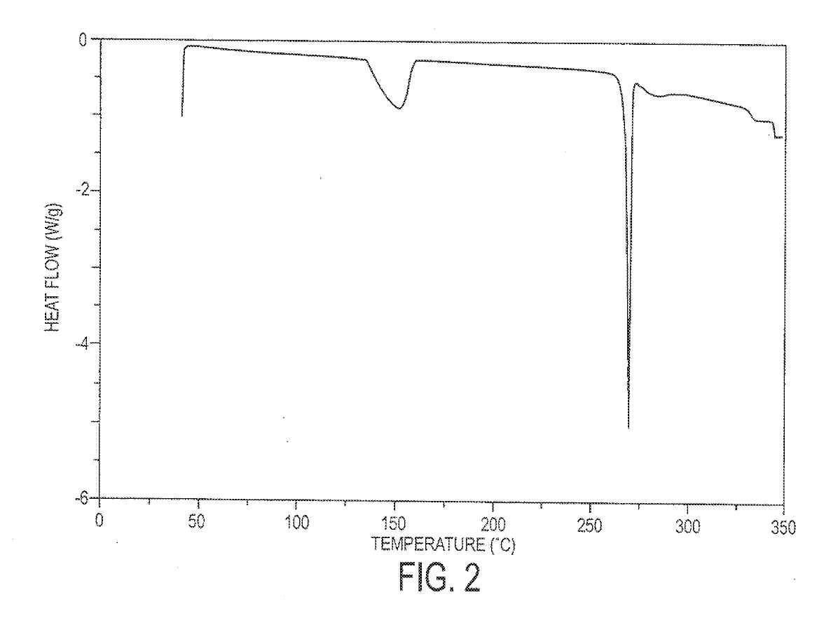

(d) A DSC curve substantially in accordance with FIG. 2.

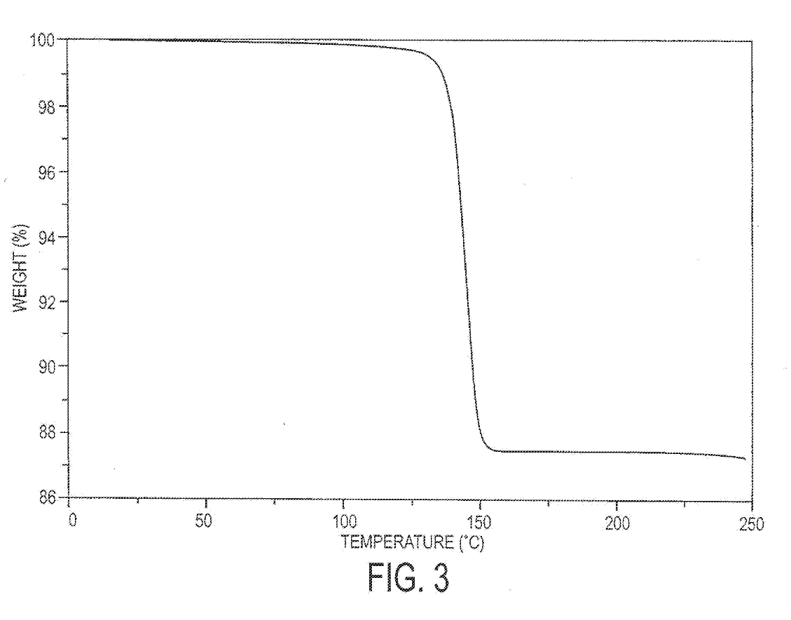

(e) A TGA curve corresponding to a weight loss of 12 to 15% w/w, substantially in accordance with FIG. 3.

In an embodiment, the present invention provides a process for preparing a dimethylformamide solvate of lenalidomide, comprising:

a) providing a solution of a methanesulfonate salt of lenalidomide in a solvent;

b) combining a base with the solution; and

c) isolating a dimethylformamide solvate of lenalidomide.

The individual steps are separately described hereinbelow.

Step a) involves providing a solution of a methanesulfonate salt of lenalidomide. The solution of methanesulfonate salt of lenalidomide can be obtained by the dissolution of a methanesulfonate salt of lenalidomide in a suitable solvent, or it may be obtained from a previous processing step where the methanesulfonate salt of lenalidomide is formed in solution. Any form of methanesulfonate of lenalidomide is acceptable for preparing a solution, such as a crystalline or amorphous form.

Suitable solvents for the dissolution include, but are not limited to, dimethylformamide (DMF) alone or in combination with a alcohol such as, for example, methanol, ethanol, isopropyl alcohol, n-butanol and the like. The amount of DMF in a solvent mixture may range from about 5% to about 100%, by volume.

The solution may be prepared at any temperatures up to the boiling point of the solvent, which may range from about 20° C. to about 180° C.

The solution may be optionally treated with an agent such as activated charcoal to enhance the color of the compound, then typically will be filtered through an inert medium, such as through a bed of flux calcined diatomaceous earth (e.g., HYFLO), to remove the carbon.

The solution may optionally be filtered by passing through paper, glass fiber, or other membrane material, or a bed of a clarifying agent, such as CELITE. Depending upon the equipment used and the concentration and temperature of the solution, the filtration apparatus may need to be heated to avoid premature crystallization.

Step b) involves combining a base with the solution from a).

The base may be added to the solution of step a) and optionally the obtained reaction mass may be subjected to cooling, and/or adding an anti-solvent, to initiate and enhance the formation of the solvate of lenalidomide.

Suitable bases include, but are not limited to, triethylamine, methylamine, pyridine, isopropylamine, diisopropylethylamine, imidazole, N-methylmorpholine, ammonia, and the like. The quantity of base used may range from about 0.5 to about 2 molar equivalents, or about 1 molar equivalent, per equivalent of methanesulfonate salt of lenalidomide.

The exact cooling temperatures and times required for a desired extent of precipitation of solid can be readily determined by a person skilled in the art.

For example, the solution can be cooled to below 40° C. and stirred for about 15 minutes to 2 hours, or longer, to initiate and enhance the crystallization. Temperatures may range from about 0° C. to about 40° C. Suitable anti-solvents that can be used to initiate precipitation include but are not limited to alcohols, such as, for example, methanol, ethanol, isopropanol, n-butanol, and the like.

Step c) involves isolating the DMF solvate of lenalidomide.

The DMF solvate of lenalidomide may be isolated from the reaction mixture by general techniques known to one skilled in the art, and may utilize conventional, suitable methods to recover the solid from the mixture. For example, the DMF solvate of lenalidomide may be isolated using techniques such as, for example, filtration by gravity or by suction, centrifugation, decantation, and the like. After isolation, the solid may optionally be washed with a suitable solvent such as DMF, optionally in combination with a co-solvent, when desired.

In another embodiment, the present invention provides a process for preparing a dimethylsulfoxide solvate of lenalidomide, the process comprising similar steps as were mentioned above for the dimethylformamide solvate of lenalidomide. In those steps, dimethylsulfoxide will be substituted for N,N-dimethylformamide.

A dimethylsulfoxide solvate of lenalidomide obtained by the process of the present invention may be characterized by any one or more of:

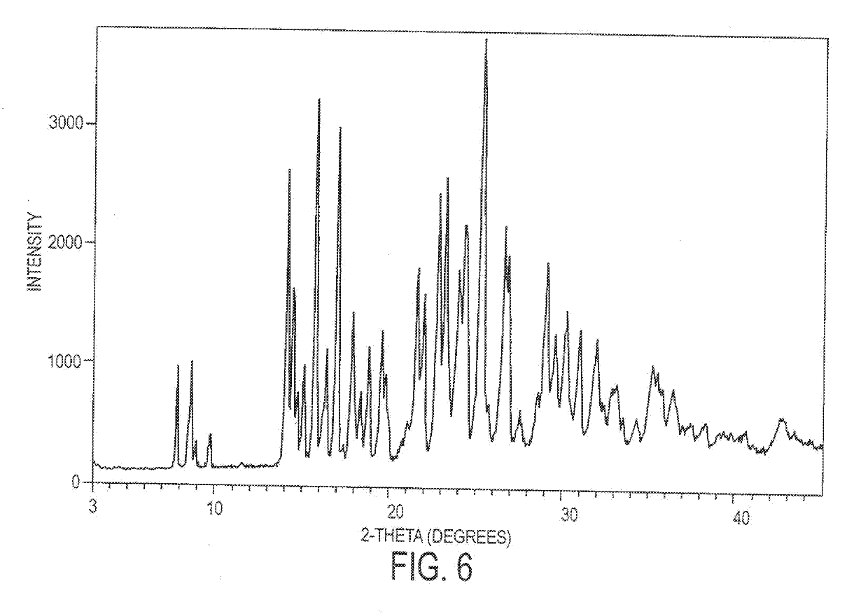

a) An XRPD pattern substantially in accordance with FIG. 6.

b) An XRPD pattern having peaks at about 7.7, 8.8, 14.0, 14.6, 15.5, 15.9, 16.4, 17.4, 18.7, 19.5, 20.3, 21.0, 21.9, 22.3, 23.6, 24.6, 25.3, and 27.9, ±0.2 degrees 2-theta.

c) An XRPD pattern having characteristic peaks at about 14.6, 17.4, 21.9, and 25.3, ±0.2 degrees 2-theta.

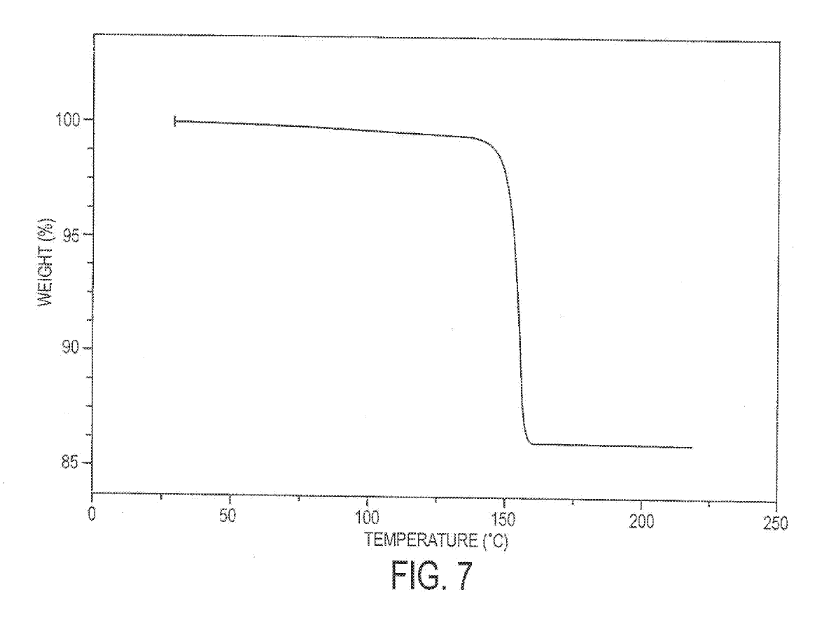

d) A TGA curve corresponding to a weight loss of 12 to 18% w/w, substantially in accordance with FIG. 7.

In yet another aspect, the present invention provides another process for preparing a solvate of lenalidomide, comprising:

a) providing a solution of lenalidomide in a solvent; and

b) isolating a solid solvate of lenalidomide.

The individual steps are separately described hereinbelow.

Step a) involves providing a solution of lenalidomide. The solution of lenalidomide can be obtained by dissolving lenalidomide in a solvent, or it can be obtained from a previous processing step where lenalidomide is formed. Any form of lenalidomide is acceptable for forming a solution, such as any crystalline or amorphous form. Lenalidomide may be obtained by any process, for example by a process disclosed in Indian Patent Application No. 47/CHE/2006, or it can be obtained by a process disclosed in Indian Patent Application No. 1422/CHE/2008.

The solution of lenalidomide may be obtained in a single solvent or in a combination of co-solvents. Useful solvents for preparing solvate forms include dimethylformamide, dimethylsulfoxide, and dimethylacetamide.

A co-solvent may be, for example: a hydrocarbon such as toluene, xylene, n-hexane, n-heptane, and cyclohexane; an ester, such as, for example, ethyl acetate, n-propyl acetate, n-butyl acetate, and t-butyl acetate; an alcohol, such as, for example, methanol, ethanol, and isopropyl alcohol; an ether, such as, for example, 1,4-dioxane, diethyl ether, tetrahydrofuran, diisopropyl ether, and methyl t-butyl ether; a nitrile solvent, such as, for example, acetonitrile and propionitrile; a halogenated solvent, such as, for example, dichloromethane, ethylene dichloride, and chloroform; a ketone, such as, for example, acetone, ethyl methyl ketone, methyl isobutyl ketone, and the like; and mixtures thereof or their combinations with water in various proportions. The amount of solvent in a solvent mixture may range from about 5% to about 100%, by volume.

The concentration of lenalidomide in a solvent or solvent mixture is not critical as long as sufficient solvent is employed to ensure total dissolution. However, the amount of solvent employed is ordinarily kept as low as possible to avoid excessive product losses during crystallization and isolation.

The solution can be prepared at any temperatures up to the boiling point of the solvent, which may range from about 40° C. to about 180° C.

The solution can be optionally treated with an agent such as activated charcoal to enhance the color of the compound, and then typically is filtered through an inert medium such as through a bed of flux calcined diatomaceous earth (e.g., HYFLO), to remove the carbon.

The solution can optionally be filtered by passing through paper, glass fiber, or other membrane material, or a bed of a clarifying agent, such as CELITE. Depending upon the equipment used and the concentration and temperature of the solution, the filtration apparatus may need to be heated to avoid premature crystallization.

Step b) involves isolating a solid solvate. The isolation of solid may be effected by precipitation/crystallization from the solution. The process of precipitation/crystallization may include methods such as cooling for a sufficient period of time to effect crystallization, partial removal of the solvent from the mixture, seeding, adding an anti-solvent to the solution, or a combination thereof.

For example, the solid may be isolated by cooling the solution for a period of about 30 minutes to 2 hours, or longer, to initiate and enhance the crystallization. Suitable temperature may range from about 0° C. to about 50° C.

For example, the solid can be isolated by cooling below 20° C. for a period of about 1 to about 20 hours, or longer.

The solvated form of lenalidomide separates from the solution and one skilled in the art may utilize conventional, suitable methods to recover the solid from the mixture. For example, the solvated form of lenalidomide may be recovered by using any technique, such as, for example, filtration by gravity or by suction, centrifugation, decantation, and the like. After solvate recovery, the solid may optionally be washed with suitable solvent, such as DMF or DMSO, optionally in combination with a co-solvent, as desired.

In one embodiment, the present invention also includes a process for preparing a solvate of lenalidomide, which process comprises the step of slurrying lenalidomide in a solvent mixture of an organic solvent and a co-solvent, for a time sufficient to crystallize a solvate of lenalidomide.

Organic solvent suitable for use in this embodiment include dimethylformamide, dimethylacetamide, dimethyl sulfoxide, and mixtures thereof. Suitable co-solvents include those in the list of co-solvents given above.

The slurrying may be performed at temperatures about 20-40° C., or higher, for a sufficient period of time, such as about 1 to about 5 hours, or longer.

Optionally, the obtained solvated forms of lenalidomide may be dried. The drying of solid material may be carried out under suitable conditions to afford the desired solvate of lenalidomide, containing a desired amount of residual solvents.

The drying may be carried out at reduced pressures, such as below about 650 mm Hg, or below about 50 mm Hg, at temperatures such as about 35° C. to about 70° C. The drying may be carried out for any desired time period that achieves the desired result, such as times about 1 to 20 hours, or longer. Drying may also be carried out for shorter or longer periods of time depending on the product specifications.

Drying may be suitably carried out using any equipment such as a rotary dryer, tray dryer, vacuum oven, air oven, or using a fluidized bed dryer, spin flash dryer, flash dryer, and the like.

The solvate of lenalidomide obtained from any described process may be utilized for the preparation of amorphous as well as crystalline polymorphs.

In another embodiment, the present application provides pharmaceutical compositions comprising a solvate of lenalidomide and at least one pharmaceutically acceptable excipient.

Lenalidomide solvates obtained by the processes of the present invention may optionally be converted to desired crystalline or amorphous forms of lenalidomide.

Lenalidomide and its impurities may be analyzed in materials containing lenalidomide and its solvates using HPLC, for example using the following set of conditions:

Instrument: Waters 2695 separation module with 2996 PDA detector.

Column: 250×4.6 mm, 5 μm (Waters Xterra RP-18).

Buffer: 1.36 g of potassium dihydrogen orthophosphate anhydrous is dissolved in 100 mL of milli-Q water, pH of the solution is adjusted to 3.5±0.05 using dilute phosphoric acid, and the solution is filtered through a 0.45 μm membrane filter.

Mobile Phase A: Buffer.

Mobile Phase B: Filtered and degassed mixture of methanol and acetonitrile in the ratio of 90:10 by volume.

Flow rate: 1.0 mL/minute.

Wavelength of detection: 210 nm.

Column temperature: Ambient.

Injection volume: 10 μL.

Run lime: 60 minutes.

Diluent: Mobile phase A and mobile phase B (1:1 by volume).

Gradient program:

| Time (minute) | Mobile Phase A | Mobile Phase B |
|---|---|---|
| 0 | 90 | 10 |
| 15 | 90 | 10 |
| 40 | 45 | 55 |
| 52 | 45 | 55 |
| 53 | 90 | 10 |
| 60 | 90 | 10 |

A sample is prepared for analysis by placing an accurately weighed amount that contains about 50 mg of lenalidomide into a 50 mL volumetric flask, dissolving the lenalidomide content in diluent solution, and diluting to volume with the diluent. A portion can be filtered before injection into the chromatograph.

The same method may also be utilized for analyzing the purity of lenalidomide solvates and salts, including a methanesulfonate salt of lenalidomide.

In embodiments, lenalidomide obtained by the processes of the invention contains less than about 0.1% by weight, as determined using HPLC, of any of the individual impurities listed in Table 1.

TABLE 1

| Impurity | Structure | RRT* |
|---|---|---|
| A | 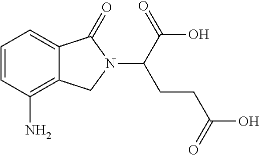 [SMILES: Nc1cccc2c1CN(C(CCC(=O)O)C(=O)O)C2=O] | 0.66 |
| B | 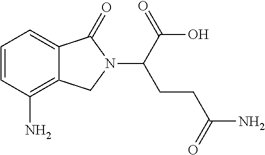 [SMILES: NC(=O)CCC(C(=O)O)N1Cc2c(N)cccc2C1=O] | 0.8 |
| C | Undetermined | 1.74 |
| D | 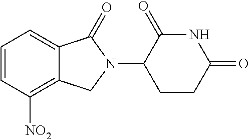 [SMILES: O=C1CCC(N2Cc3c(cccc3[N+](=O)[O-])C2=O)C(=O)N1] | 1.98 |

*Relative retention time, lenalidomide = 1.

In an embodiment, the present invention provides lenalidomide having a purity greater than about 99.8% by weight, using HPLC analysis.

In an embodiment, the present invention provides lenalidomide having a purity greater than about 99.8% by weight and containing less than about 0.1% by weight of impurity C, by HPLC.

A sample of the commercially available pharmaceutical product REVLIMID® has been analyzed and the purity of the contained lenalidomide was 99.76% by weight. Further, the sample analysis showed that significant concentrations of impurities were present in the sample, the results being shown in Table 2.

TABLE 2

| Impurity | Structure | Area-% |
|---|---|---|
| Impurity A | 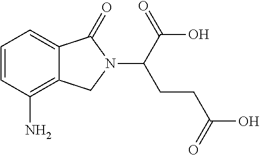 [SMILES: Nc1cccc2c1CN(C(CCC(=O)O)C(=O)O)C2=O] | 0.03 |
| Impurity B | 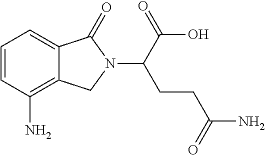 [SMILES: NC(=O)CCC(C(=O)O)N1Cc2c(N)cccc2C1=O] | 0.02 |
| Impurity C | Undetermined | 0.18 |

The XRPD data reported in this application were obtained using copper Kα radiation, having the wavelength 1.5418 Å, and were obtained using a Bruker AXS D8 Advance Powder X-ray Diffractometer.

Differential scanning calorimetric analysis was carried out in a DSC Q1000 model from TA Instruments with a ramp of 5° C./minute with a modulation time of 60 seconds and a modulation temperature of ±1° C. The starting temperature was 0° C. and ending temperature was 200° C.

Certain specific aspects and embodiments of the processes of the present invention will be explained in more detail with reference to the following examples, which are provided for purposes of illustration only and should not be construed as limiting the scope of the invention in any manner. In the examples, percentages are expressed by weight unless the context indicates otherwise.

### Example 1

### Preparation of a Methanesulfonate Salt of Lenalidomide

3-(4-nitro-1-oxo 1,3-dihydroisoindol-2-yl)piperidine-2,6-dione (10 g), methanol (300 mL), 10% palladium on carbon (0.3 g) and methanesulfonic acid (4.5 mL; d:1.48) are charged into a conical flask and then transferred into an autoclave. Hydrogen gas (90 psi) is applied to the suspension at 30° C. and stirred for 3-4 hours. The reaction mixture is filtered through a celite bed and the bed washed with methanol (20 mL). The obtained filtrate is concentrated to a volume of 100 mL and stirred for 20 minutes. The reaction mass is filtered and the solid dried for 4 hours at 50° C. to give 8 g of a methanesulfonate salt of lenalidomide.

Purity: 99.87% by HPLC.

Impurity A: 0.01%; Impurity B: 0.01%; Impurity C, 0.04%; Impurity D: not detected (ND).

### Example 2

### Preparation of a DMF Solvate of Lenalidomide

A methanesulfonate salt of lenalidomide from Example 1 (2 g), a mixture of solvents N,N-dimethylformamide (DMF) and methanol (1:2 by volume, 18 mL) and triethylamine (0.8 mL) are charged into a round bottom flask and stirred for 2 hours at 25 to 35° C. The suspension is filtered and washed with a mixture of DMF and methanol (1:2 by volume, 4 mL). The obtained solid is dried at 50° C. for 4 hours to afford 1.25 g of title compound.

Purity: 99.96% by HPLC.

Impurity A: ND; Impurity B: 0.01%; Impurity C: 0.01%; Impurity D: ND.

XRPD: substantially in accordance with FIG. 1.

DSC: substantially in accordance with FIG. 2,

TGA: 12.35% w/w loss as represented in FIG. 3.

### Example 3

### Preparation of a DMF Solvate of Lenalidomide

A methanesulfonate salt of lenalidomide from Example 1 (2 g) and a solvent mixture of DMF and methanol (1:2 by volume, 20 mL) are charged into a round bottom flask and stirred for 10 minutes at 25-30° C. The solution is decanted into another vessel and triethylamine (0.8 mL) is added and stirred for 2 hours. The mixture is filtered and the solid washed with the solvent mixture of DMF and methanol (1:2 by volume, 4 mL). The solid is dried at 50° C. for 4 hours to afford 1.1 g of title compound.

Purity: 99.96% by HPLC.

Impurity A: 0.01%; Impurity B: 0.01%; Impurity C: 0.01%; Impurity D: ND.

### Example 4

### Preparation of a DMF Solvate of Lenalidomide

A methanesulfonate salt of lenalidomide (1 g) is dissolved in DMF (3 mL) at 25-35° C., and triethylamine (0.4 mL), and methanol (6 mL) are added. The suspension is stirred for 2 hours, filtered and the obtained solid washed with methanol (2 mL). The resultant solid is dried for 4-5 hours at a temperature of 50° C. under reduced pressure to afford 0.71 g of title compound.

Purity: 99.86% by HPLC.

Impurity A: 0.02%; Impurity B: ND; Impurity C: ND; Impurity D: 0.04%.

### Example 5

### Preparation of a DMF Solvate of Lenalidomide

A methanesulfonate salt of lenalidomide (5 g) is dissolved in DMF (15 mL) and triethylamine (1.96 mL) and the clear solution is stirred for 2 hours at a temperature of 25-35° C. The suspension is filtered and washed with DMF (1 mL) and the solid dried for 4 hours at a temperature of 48° C. to afford 3.2 g of title compound.

Purity: 99.83% by HPLC

Impurity A: 0.01%; Impurity B: 0.01%; Impurity C: ND; Impurity D: ND.

### Example 6

### Preparation of a DMF Solvate of Lenalidomide

Lenalidomide (0.5 g) is dissolved in DMF (10 mL) at room temperature and stirred for 10-15 minutes. Toluene (80 mL) is charged to the solution and stirred for 25 minutes. The obtained suspension is filtered and the solid dried under vacuum at 25 to 35° C. for 1 hour, to afford 0.42 g of title compound.

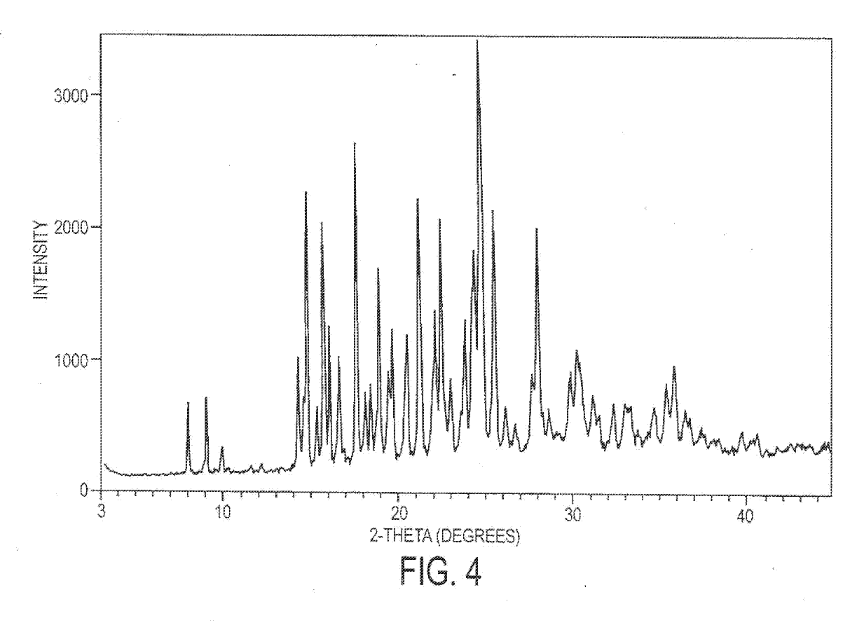

XRPD: substantially in accordance with FIG. 4.

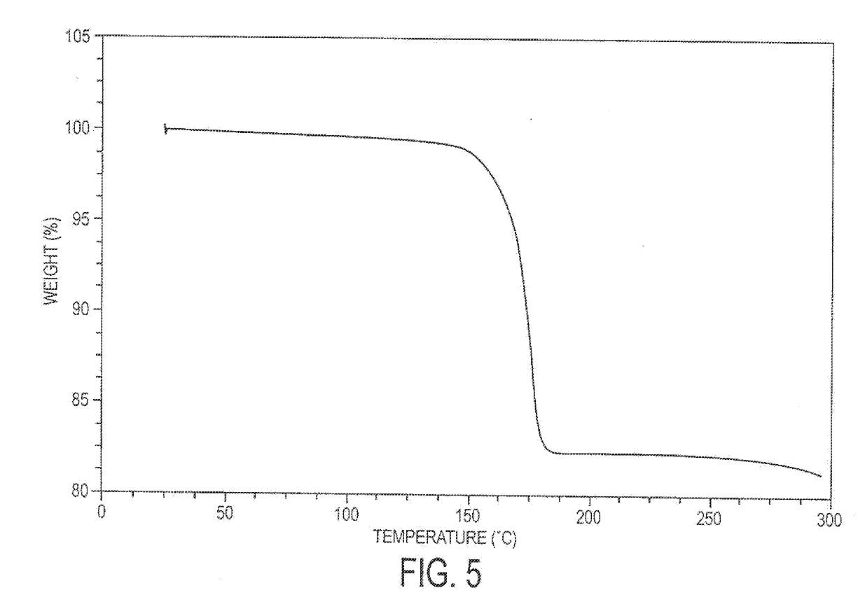

TGA: 13.92% weight loss as represented in FIG. 5.

### Example 7

### Preparation of a DMF Solvate of Lenalidomide

Lenalidomide (1 g) is dissolved in DMF (7 mL) at room temperature to produce a clear solution. The solution was maintained at ambient temperature for about 2-3 hours resulting in suspension. The suspension was filtered and the solid was dried under vacuum at 25 to 35° C. for 1-2 hours, to afford 0.4 g of title compound.

TGA: 12.98% weight loss.

### Example 8

### Preparation of a DMF Solvate of Lenalidomide

Lenalidomide (0.5 g) is dissolved in a 1:1 by volume solvent mixture of DMF and toluene (15 mL) at a temperature of 125° C. and then filtered. The filtrate is maintained below 10° C. for a period of 20 hours. The suspension is filtered and the solid dried under vacuum at a temperature of 25 to 35° C. for a period of 1 hour, to afford 0.37 g of the title compound.

TGA: 13.32% weight loss.

### Example 9

### Preparation of a DMF Solvate of Lenalidomide

Lenalidomide (0.5 g) is suspended in a mixture of DMF (5 mL) and toluene (20 mL) at room temperature and stirred for 2-3 hours. The suspension is filtered and the solid dried at 30° C. under vacuum for 1 hour, to obtain 0.39 g of the title compound.

TGA: 12.28% weight loss.

### Example 10

### Preparation of a DMSO Solvate of Lenalidomide

Lenalidomide (0.5 g) is dissolved in dimethylsulfoxide (DMSO, 10 mL) at room temperature and stirred for 10 minutes. Toluene (60 ml) is charged to the solution and stirred for 30 minutes. The produced suspension is filtered and the solid dried under vacuum at 25 to 35° C. for 1 hour, to afford 0.4 g of title compound.

XRPD: substantially in accordance with FIG. 6.

TGA: 17.69% weight loss, as represented in FIG. 7.

### Example 11

### Preparation of a DMSO Solvate of Lenalidomide

Lenalidomide (1.1 g) is dissolved in DMSO (3 mL) at room temperature and stirred for 2-3 hours. The produced suspension is filtered and the solid dried under vacuum at 25 to 35° C. for 1 hour, to afford 0.17 g of the title compound.

TGA: 13.83% weight loss.

### Example 12

### Preparation of a DMSO Solvate of Lenalidomide

Lenalidomide (0.5 g) is dissolved in a solvent mixture of DMSO and toluene (1:10 by volume, 55 mL) at a temperature of 125° C., and then filtered. The filtrate is maintained below 10° C. for a period of 20 hours. The produced suspension is filtered and the solid dried under vacuum at a temperature of 25 to 35° C. for a period of 1 hour, to afford 0.37 g of the title compound.

TGA: 16.09% weight loss.

### Example 13

### Preparation of a DMSO Solvate of Lenalidomide

Lenalidomide (0.5 g) is suspended in a mixture of DMSO (3 mL) and toluene (20 mL) at room temperature and stirred for 2-3 hours. The suspension is filtered and the solid dried at 30° C. under vacuum for 1 hour, to obtain 0.4 g of the title compound.

TGA: 14.37% weight loss.

1-20. (canceled)

21. A process for preparing a N,N-dimethylformamide solvate of Lenalidomide, comprising:
a) reducing 3-(4-nitro-1-oxo 1,3-dihydroisoindol-2-yl)piperidine-2,6-dione of formula II, 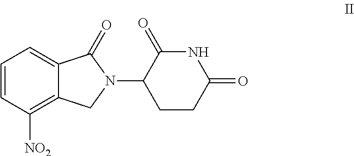 [SMILES: O=C1CCC(N2Cc3c(cccc3[N+](=O)[O-])C2=O)C(=O)N1]
with a catalyst in the presence of an acid to give an acid addition salt of lenalidomide of formula III, 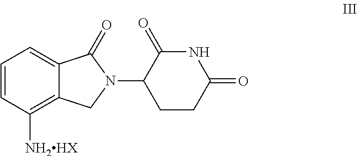
wherein HX is an acid; and
b) reacting the acid addition salt of lenalidomide of formula III with a base in the presence of N,N-dimethylformamide to provide a N,N-dimethylformamide solvate of lenalidomide.

22. The process of claim 21, wherein the catalyst in step a) is selected from a metal catalyst or a chemical reducing agent.

23. The process of claim 21, wherein the reduction of formula II is carried out in the presence of a hydrogen source.

24. The process of claim 21, wherein the acid used in step a) is selected from an organic acid or an inorganic acid.

25. The process of claim 21, wherein the reduction of formula II is carried out in the presence of a solvent selected from water, an alcohol solvent, a ketone solvent, dimethyl formamide, N,N-dimethyl acetamide, dimethyl sulfoxide or mixture thereof.

26. The process of claim 21, wherein an acid addition salt of lenalidomide of formula III is an alkyl- or aryl-sulfonate salt of lenalidomide.

27. The process of claim 21, wherein an acid addition salt of lenalidomide of formula III is a methanesulfonate salt of lenalidomide.

28. The process of claim 21, wherein the base used in step b) is selected from an organic base or an inorganic base.

29. The process of claim 28, wherein the base is an alkylamine.

30. The process of claim 21, further comprising converting N,N-dimethylformamide solvate of lenalidomide to lenalidomide.

31. A process for preparing a N,N-dimethylformamide solvate of lenalidomide, comprising:
a) providing a solution of an acid addition salt of lenalidomide of formula III in a solvent comprising N,N-dimethylformamide;
b) combining a base with the solution of step a);
c) optionally, adding an anti-solvent; and
d) isolating an N,N-dimethylformamide solvate of lenalidomide.

32. The process of claim 31, wherein an acid addition salt of lenalidomide of formula III is a methanesulfonate salt of lenalidomide.

33. The process of claim 31, wherein the solvent in step a) contains a co-solvent selected from an alcohol such as methanol, ethanol, isopropyl alcohol, n-butanol and the like.

34. The process of claim 31, wherein the base in step b) is an alkylamine.

35. The process of claim 31, wherein in the anti-solvent is an alcohol solvent.

36. The process of claim 31, wherein step b) or c) further comprises maintaining the solution or suspension at a temperature below 40° C.

37. A process for preparing a N,N-dimethylformamide solvate of lenalidomide, comprising:
a) providing a solution or suspension of lenalidomide in a solvent comprising N,N-dimethylformamide;
b) optionally, adding a solvent selected from an alcohol or a hydrocarbon;
c) maintaining the solution or suspension at a temperature below 40° C.;
d) isolating an N,N-dimethylformamide solvate of lenalidomide.

38. The process of claim 37, wherein the solvent in step a) further contains a co-solvent selected from an alcohol such as methanol, ethanol, isopropyl alcohol, and n-butanol or a hydrocarbon solvent selected from toluene, xylene, hexane, heptane, and cyclohexane.

39. A N,N-dimethylformamide solvate of lenalidomide characterized by an X-ray powder diffraction pattern having peaks at 17.0, 23.3 and 25.4±0.2 degrees 2-theta.

40. The N,N-dimethylformamide solvate of lenalidomide of claim 32, further characterized by an X-ray powder diffraction pattern as depicted in FIG. 1.
